(19) United States
(12) Patent Application Publication
(10) Pub. No.: US 2004/0038247 A1
(43) Pub. Date: Feb. 26, 2004

(54) Nucleic acid constructs including a novel t-cell active promoters, and pharmaceutical compositions and methods utilizing same for regulating t-cell mediated immune response

(76) Inventors: Yin Hwee Tan, Vancouver, CA (US); Sydney Brenner, Cambs (GB); Byrappa Venkatesh, College Green (SG)

Correspondence Address: WILDMAN, HARROLD, ALLEN & DIXON, 225 WEST WACKER DRIVE, CHICAGO, IL, 60606, US

(21) Appl. No.: 10/362,010

(22) PCT Filed: Aug. 16, 2001
(86) PCT No.: PCT/IL01/00765

(51) Int. Cl. C12Q 1/68; C07H 21/04; C12N 9/22; C12N 5/08

(52) U.S. Cl. 435/6; 435/69.1; 435/320.1; 435/372; 435/199; 536/23.2

(57) ABSTRACT

An isolated nucleic acid is disclosed, including a promoter sequence being transcriptionally functional in a T-lymphocyte undergoing activation and transcriptionally less functional in the T-lymphocyte prior to the activation.

### FIELD AND BACKGROUND OF THE INVENTION

The present invention relates to an isolated nucleic acid sequence including a promoter functional only in T-lymphocytes undergoing activation and to nucleic acid constructs, pharmaceutical compositions and methods of utilizing same. More particularly, the present invention relates to nucleic acid constructs capable of, for example, expressing exogenous polynucleotides in T-cells undergoing activation and to the use of such constructs in pharmaceutical compositions and methods designed for regulating T-cell mediated immune response in mammals.

An immune response in mammals is a complex event involving numerous cellular and molecular constituents.

T-cells play a central role in the immune response as effectors and regulators, coupling antigen recognition and the transmission of activation signals. The diverse responses of T-cells are referred to as cell-mediated immune reactions and are initiated by recognition of antigens via the T-cell receptor (TCR).

The T-cell repertoire recognizes a broad range of antigens. As with B cells, a particular clonal line of T-cells can only recognize a single antigen. However, unlike B cells, T-cells recognize antigens only when they are presented to the TCR as peptides in a complex with major histocompatibility molecules (MHC). The TCR on most T-cells consist of immunoglobulin-like integral membrane glycoproteins containing 2 polypeptide subunits, alpha and beta, of similar molecular weight. Each T-cell receptor subunit has an external amino terminal containing a variable (V) domain joined to a constant (C) domain extending intracellularly. The genes for the T-cell receptor subunits are constructed through somatic rearrangement of different gene segments, of which there are at least 3 types for the alpha; V, joining (J) and C domains, and at least 4 types for the beta; V, diversity (D), J and C domains. An invariant CD3 complex of polypeptides and a disulfide-linked homodimer are noncovalently associated with the TCR. These associated polypeptides are responsible for the signal transduction functions of the TCR and are also required for efficient surface expression of the receptor.

Interaction of antigen in the proper MHC context with the TCR leads to a regulated series of events resulting in differentiation, proliferation, and the acquisition of T-cell immunologic function.

This functional response which is termed T-cell activation, depends on whether the T-cell receives co-stimulatory signals through other surface receptors and which signal transduction pathways are activated (Klausner, R. D. and Samelson, L., E. (1991) Cell 64: 875-878; Clevers, H. et al. (1988) Annu. Rev. Immunol. 6: 629-662).

The signaling cascades initiated by TCR activation include the inositol tri-phosphate/Ca²⁺, diacylglycerol/protein kinase C, Ras/mitogen-activated protein kinase, and the PI 3-K pathways. Components of these pathways transmit information into the nucleus to activate the genes that code for a variety of secreted factors, such as IL-2, IL-4, IL-7, IL-9, IL-10, and interferon-γ, which subsequently induce the proliferation, maturation, and function of cellular components of the immune system. These factors can stimulate the induction of antibody secretion and class switching in B cells, the regulation of humoral immunity, and induction of tumoricidal and inflammatory activities (Alderson, M., R., et al (1991) J. Exp. Med. 173: 923-930; and Weiss, A. (1991) Annu. Rev. Genet. 25:487-510).

The TCR antigen repertoire is established by developmentally regulated TCR gene rearrangements and is shaped by intrathymic selection processes. Immature T-cells undergo a selection and differentiation process based on antigen binding prior to leaving the thymus. Those that bind self-antigens while still in the cortex of the thymus are eliminated by apoptosis, establishing immunological tolerance. Failure to eliminate auto-reactive populations of cells has been shown to result in autoimmune disease.

Defects in TCR genes, TCR expression, and T-cell subtype population levels have been noted in lymphomas, leukemias, allergic responses and autoimmune and immunodeficiency disorders. In transgenic mice deficient in the TCR alpha and beta subunits, B cells expand, differentiate and secrete copious amounts of antibodies that are reactive towards self antigens. Graft-versus-host disease is the result of normal T-cell responses to foreign cells, as is the cytolytic destruction of virus-infected and tumor cells. (Yui, K. et al (1992) Eur J Immunol 22: 1693-1700; Olive, C., supra (1995); Mombaerts, P., et al (1993) Cell 75: 274-282; Wen, L., et al (1994) Nature 369 :654-658: Leiden, J., M., et al. (1986) Immunogenet. 24: 17-23: and Barber, D., F., and Lopez de Castro, J., A., Genbank accession L34734)

Thus, the molecular elucidation of T-cell activation and of T-cell mediated immune responses is of utmost importance in the diagnosis, prevention and treatment of various immune related disorders.

As part of the present study, the present inventors have investigated genomic sequences from the Japanese pufferfish, Fugu rubripes.

Unexpectedly, it was uncovered that Fugu, which evolved from the common ancestor of fish and of mammals such as humans and mice approximately 400 million years ago, contains a genetic regulatory sequence element that can regulate the expression of the lymphocyte specific src family protein tyrosine kinase Lck, critical for T-cell activation, in the mammalian immune system as effectively as its mammalian equivalent.

While reducing the present invention to practice, a promoter sequence which targets and regulates the expression of T-lymphocyte specific Lck in the thymus, testis and to a lesser extent the whole blood of a mouse was isolated. This sequence, which spans one to several kilobases is contiguous and smaller than the human Lck promoter which spans 34 kilobases of DNA (Wildin, R. S. et al., Developmental Regulation of Lck Gene Expression in T-Lymphocytes (1991) J. Exp. Med. 173:383-393). Thus, in contrast to its mammalian counterpart the Fugu derived sequence can be effectively utilized to target the expression of exogenous polynucleotides in T-cells for the purposes of regulating T-cell mediated immune response in mammals.

### SUMMARY OF THE INVENTION

According to one aspect of the present invention there is provided an isolated nucleic acid comprising a promoter sequence being transcriptionally functional in a T-lymphocyte undergoing activation and transcriptionally less functional in the T-lymphocyte prior to the activation.

According to still further features in the described preferred embodiments there is provided a nucleic acid construct comprising the promoter sequence described above.

According to still further features in the described preferred embodiments the nucleic acid construct described above further comprising an additional polynucleotide sequence being under the transcriptional control of the promoter sequence.

According to still further features in the described preferred embodiments the nucleic acid construct further comprising a positive and/or a negative selection markers.

According to still further features in the described preferred embodiments there is provided a host cell or animal comprising the nucleic acid construct described above.

According to still further features in the described preferred embodiments there is provided a pharmaceutical composition comprising an effective amount of the nucleic acid construct described hereinabove and a pharmaceutically acceptable carrier.

According to another aspect of the present invention there is provided a method of identifying and/or isolating T-cells undergoing activation from a population of cells, the method comprising the steps of: (a) transforming the population of cells with a nucleic acid construct including a polynucleotide encoding a reporter molecule being under the transcriptional control of a promoter sequence being transcriptionally functional in T-cells undergoing activation and transcriptionally less functional prior to the activation; and (b) identifying and/or isolating cells from the population of cells expressing the reporter molecule above a predetermined background value to thereby identify and/or isolate T-cells undergoing activation.

According to still further features in the described preferred embodiments the reporter molecule is an RNA molecule or a polypeptide molecule.

According to still further features in the described preferred embodiments the polypeptide molecule is selected from the group consisting of an enzyme, a ligand and a fluorophore.

According to yet another aspect of the present invention there is provided a method of eliminating T-cells undergoing activation from a population of cells, the method comprising the step of transforming a population of cells including the T-cells undergoing activation with a nucleic acid construct including a polynucleotide encoding a cytotoxic molecule being under the transcriptional control of a promoter sequence being transcriptionally functional in the T-cells undergoing activation and transcriptionally less functional prior to the activation to thereby eliminate the T-cells undergoing activation from the population of cells.

According to still further features in the described preferred embodiments the cytotoxic molecule is an RNA molecule or a polypeptide molecule.

According to still further features in the described preferred embodiments the RNA molecule is a ribozyme or an anti-sense RNA molecule.

According to still further features in the described preferred embodiments the polypeptide molecule is an enzyme or a ligand.

According to still another aspect of the present invention there is provided a method of enhancing T-cell activation, the method comprising the step of transforming a population of cells including the T-cells undergoing activation with a nucleic acid construct including a polynucleotide encoding a cell cytokine capable of enhancing T-cell activation and being under the transcriptional control of a promoter sequence being transcriptionally functional in T-cells undergoing activation and transcriptionally less functional prior to the activation to thereby enhance T-cell activation.

According to still further features in the described preferred embodiments the cell cytokine is selected from the group consisting of IL-2, IL-4, IL-7, IL-9, IL-10 and interferon-γ.

According to an additional aspect of the present invention there is provided a method of suppressing T-cell activation, the method comprising the step of transforming a population of cells including T-cells undergoing activation with a nucleic acid construct including a polynucleotide encoding a molecule capable of disrupting a signaling cascade initiated by the T-cell activation, the polynucleotide being under the transcriptional control of a promoter sequence being transcriptionally functional in T-cells undergoing activation and transcriptionally less functional prior to the activation to thereby suppress T-cell activation.

According to an additional aspect of the present invention there is provided a method of identifying a promoter specific regulatory factor, the method comprising the steps of (a) providing a reporter construct including a reporter molecule being under the expression control of a promoter sequence being transcriptionally functional in a T-lymphocyte undergoing activation and transcriptionally less functional in the T-lymphocyte prior to the activation; (b) incubating the reporter construct with a candidate regulatory factor under conditions suitable for transcription and optionally translation of the reporter molecule; and (c) monitoring a presence of the reporter molecule to thereby determine if the candidate regulatory factor is capabale of regulating expression of the reporter molecule.

According to still further features in the described preferred embodiments the molecule capable of disrupting a signaling cascade initiated by the T-cell activation is an RNA molecule or a polypeptide molecule.

According to still further features in the described preferred embodiments the RNA molecule is a ribozyme or an anti-sense RNA molecule.

According to still further features in the described preferred embodiments the polypeptide molecule is an enzyme or a ligand.

According to further features in preferred embodiments of the invention described below, the promoter sequence is at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the promoter sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

The present invention successfully addresses the shortcomings of the presently known configurations by providing an isolated nucleic acid sequence which includes a promoter sequence functional or upregulatable in T-lymphocytes undergoing activation. The present invention further provides nucleic acid constructs utilizing said promoter sequence and to methods of utilizing these nucleic acid constructs for regulating T-lymphocyte activation and for identifying, isolating or eliminating T-lymphocytes undergoing activation from a population of cells such as circulating blood cells of an individual.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a restriction map illustrating the overlap between the two cosmids (52C1 and 88A8) used in generating the contiguous 61 kb DNA fragment which includes the Fugu LCK 4 kb promoter region (SEQ ID NO:25).

FIG. 2 illustrates GFP expression levels in a Jurkat T-cell line as obtained from a construct which includes a 2 kb fragment of the Fugu LCK sequence (SEQ ID NO:24) as a promoter. Fluorescence was detected as described in the Examples section.

FIG. 3 illustrates GFP expression levels in a JurkaT-cell line as obtained from a construct which includes a 4 kb fragment of the Fugu LCK sequence as a promoter. Fluorescence was detected as described in the Examples section.

FIG. 4 illustrates GFP expression levels in a Jurkat T-cell line as obtained from a construct which includes a 6 kb fragment of the Fugu LCK sequence (SEQ ID NO:26) as a promoter; fluorescence was measured as described in the Examples section.

FIG. 5 is a Western blot illustrating EGFP expression in Jurkat cells transfected with constructs expressing EGFP from different fragments of the Fugu Lck promoter.

FIG. 6 illustrates RT-PCR quantification of the GFP expression obtained from a 2 kb or a 4 kb Fugu LCK promoter in transgenic mice.

FIG. 7 is a Northern blot illustrating transgenic mice expression of EGFP from the 4 kb Fugu Lck promoter.

FIG. 8 is a Western blot depicting EGFP expression under the regulatory control of the 2 kb (FLCK2 kb) and 0.9 kb (FLCK minimal promoter) Fugu LCK promoters in Jurkat cells. EGFP expressed under the regulatory control of the CMV promoter (CMV) in CHO, 293 and Jurkat cells was used as a positive control.

### DESCRIPTION OF THE PREFERRED EMBODIMENTS

The present invention is of nucleic acid constructs including a novel T-lymphocyte active promoter which can be utilized for regulating T-lymphocyte mediated immune response in individuals. Specifically, the present invention can be used to suppress or eliminate T-lymphocytes undergoing activation thus enabling to suppress T-lymphocyte mediated immune response in individuals suffering from immune disorders, such as, for example, an autoimmune disorder. The present invention can also be utilized to enhance T-lymphocytes undergoing activation thus enabling to enhance T-lymphocyte mediated immune response in individuals suffering, for example, from a viral infection.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The phrases “T-lymphocytes” and “T-cells” are used interchangeably herein to refer to the various T-lymphocyte subclasses including helper, suppressor and cytotoxic T-lymphocytes.

The molecular elucidation of T-cell activation and of T-cell mediated immune response is of utmost importance in the diagnosis, prevention and treatment of various immune related disorders in mammals.

As such, nucleic acid regulatory sequences which participate in the transcriptional regulation of T-cell expressed polynucleotides, during T-cell activation, are a valuable tool for studying and regulating the T-cell mediated immune response.

While reducing the present invention to practice, the present inventors have isolated a regulatory sequence element from the genome of the Japanese pufferfish (Fugu rubripes), which regulatory sequence directs the expression of the lymphocyte specific src family protein tyrosine kinase (LCK), implicated in T-cell activation, in mammalian T-lymphocytes.

This sequence, which spans anywhere from 0.9-4 kilobases is contiguous and smaller than the human LCK promoter which spans about 34 kilobases of genomic DNA. Thus, in contrast to its mammalian counterpart, the Fugu derived sequence can be effectively utilized to target the expression of exogenous polynucleotides in T-cells for the purposes of regulating T-cell mediated immune response in mammals.

Thus, according to one aspect of the present invention, there is provided an isolated nucleic acid comprising a promoter sequence being transcriptionally functional in a T-lymphocyte undergoing activation and transcriptionally less functional in the T-lymphocyte prior to the activation.

As used herein, T-cell activation refers to the regulated series of events resulting in differentiation, proliferation, and the acquisition of T-cell immunologic function.

As such, this promoter sequence is activated or upregulated in response to activation of the T-lymphocyte. Activation of a T-lymphocyte can be initiated by, for example, interaction with a foreign antigen in the case of a cytotoxic T-lymphocyte, or interaction with a B-cell in the case of helper or suppressor T-lymphocytes. For further detail on the T-cell mediated immune response in mammals please see, Abbas, K. S., Lichtman, A. H. and Pober, J. S. Cellular and Molecular Immunology. 1997. Third Edition. 494 pp. W. B. Saunders Company, Philadelphia.

According to a preferred embodiment of the present invention, the promoter sequence is at least 50%, at least 65%, at least 75%, at least 80%, at least 90%, at least 95-100% identical to SEQ ID NOs:24, 25 26 or 27 as determined using the BestFit software of the Wisconsin sequence analysis package, utilizing the Smith and Waterman algorithm, where gap weight equals 50, length weight equals 3, average match equals 10 and average mismatch equals −9.

According to another preferred embodiment of the present invention, the promoter sequence is hybridizable with SEQ ID NOs:24, 25, 26 or 27 under mild conditions

Preferably, the isolated nucleic acid according to this aspect of the present invention is hybridizable with SEQ ID NOs:24, 25, 26 or 27 under moderate to stringent hybridization conditions.

Hybridization under mild hybridization conditions is effected by a hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C. Hybridization under moderate hybridization conditions is effected by a hybridization solution containing 10% dextrane sulfate, 1 M NaCl, 1% SDS and 5×10⁶ cpm ³²P labeled probe, at 60° C., with a final wash solution of 1×SSC and 0.1% SDS and final wash at 55 to 65, preferably 60° C., whereas, hybridization under stringent hybridization conditions is effected by a hybridization solution containing 10% dextrane sulfate, 1 M NaCl, 1% SDS and 5×10⁶ cpm ³²P labeled probe, at 65° C., with a final wash solution of 0.1×SSC and 0.1% SDS and final wash at 55 to 65, preferably 60° C.

According to another preferred embodiment of the present invention, there is provided a nucleic acid construct which includes the promoter sequence of the present invention.

Preferably, this construct also includes a polynucleotide sequence positioned under the transcriptional control of the promoter sequence of the present invention, thus enabling the activation or upregulation of transcription of this polynucleotide sequence only in T-lymphocytes undergoing activation. As is further described hereinbelow, such a polynucleotide can encode, for example, a reporter molecule, a cytotoxin or a cytokine depending on the intended purpose of the nucleic acid construct.

According to another preferred embodiment of the present invention there is provided a host cell or animal comprising the nucleic acid construct described above.

Thus, the nucleic acid construct according to the present invention can be utilized to transform cells of a mammal in order to express exogenous polynucleotides in T-lymphocytes undergoing activation.

As is further described hereinbelow and in the Examples section which follows, such cells can be every cell of a mammal, such as the case with the transgenic mice described in the Examples section, blood cells either present in, or derived from, a blood of a mammal, cells of organ tissue or cells of a cell culture.

Numerous methods are known in the art for transforming mammalian cells. Such methods include, but are not limited to, direct DNA uptake techniques, and virus or liposome mediated transformation (for further detail see, for example, “Methods in Enzymology” Vol. 1-317, Academic Press). Bombardment of cell cultures or organ derived tissues with nucleic acid coated particles is also envisaged.

The above described transformation methods and others can be utilized to transform every cell type of the intended tissue, or alternatively, some of the above methods can be utilized for T-lymphocyte specific transformation. For example, T-lymphocyte specific transformation can be effected by utilizing recombinant viruses which infect only T-lymphocytes.

It will be appreciated that the above transformation methods can be utilized to generate cells which are either transiently or stably transformed with the nucleic acid construct of the present invention.

In transient transformation, the nucleic acid construct is expressed within the cell but it is not stably integrated into the genome, as such expression is maintained in the cell as long as the nucleic acid construct is present therein. In stable transformation the nucleic acid construct or an expressing portion thereof is integrated into the host cell genome and thus it is also transferred to, and expressed in, cells divided from the initially transformed cell.

In any case, regardless of the transformation method, or the diversity of cell types transformed, the use of the promoter sequence of the present invention ensures that the polynucleotide sequence positioned downstream of the promoter will only be transcribed in T-lymphocytes undergoing activation.

It will be appreciated that the nucleic acid construct of the present invention can also be delivered into cells, such as circulating blood cells, as part of a pharmaceutical composition.

Thus, according to another preferred embodiment of the present invention, there is provided a pharmaceutical composition including an active amount of the nucleic acid construct of the present invention and a pharmaceutically acceptable carrier.

Hereinafter, the phrase “pharmaceutically acceptable carrier” refer to a carrier that does not cause significant irritation to an organism and does not abrogate the biological activity and properties of the administered active compound.

The nucleic acid construct according to the present invention which constitute the “active ingredient” of the pharmaceutical composition can be administered to the individual via various administration modes.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer a preparation in a local rather than systemic manner, for example, via injection of the preparation directly into a lymph node or an organ of the individual.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredient may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the active ingredient can be formulated as tablets, pills, dragees, capsules, liquids, gels, syrups, slurries, suspensions, and the like, for oral ingestion by a patient.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredient is conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active ingredient in water-soluble form. Additionally, suspensions of the active ingredients may be prepared as. appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include. compositions wherein the active ingredients are contained in an amount effective to achieve the intended purpose. More specifically, a therapeutically effective amount means an amount of active ingredients effective to regulate activation of T-lymphocytes.

Determination of a therapeutically effective amount is well within the capability of those skilled in the art, especially in light of the detailed disclosure provided in the Examples section which follows.

For any preparation used in the methods of the invention, the therapeutically effective amount or dose can be estimated initially from in vitro and cell culture assays. For example, a dose can be formulated in animal models to achieve a desired regulation. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen according to individual patient needs. (See e.g., Fingl, et al., 1975, in “The Pharmacological Basis of Therapeutics”, Ch. 1 p.1).

Regardless of the delivery method, the nucleic acid construct of the present invention which includes the promoter sequence activatable or upregulatable in T-cells undergoing activation can be utilized to express a regulatory molecule only in T-cells undergoing activation and as such to regulate a T-lymphocyte mediated immune response in an individual.

Thus, according to another aspect of the present invention, there is provided a method of suppressing T-cell activation. The method is effected by transforming a population of cells including T-cells undergoing activation with the nucleic acid construct of the present invention constructed for expressing a molecule capable of disrupting a signaling cascade initiated by the T-cell activation. Such a molecule can be, for example, an antisense RNA or a ribozyme molecule capable of interrupting or preventing cytokine mRNA translation, or an immunogenic portion of an antibody capable of binding and thus inhibiting cellular cytokine molecules.

Since the promoter sequence of the nucleic acid construct of the present invention is only activated or upregulated in T-cells undergoing activation, expression of the molecule capable of disrupting a signaling cascade is restricted to these cells thereby enabling the selective suppression of T-lymphocytes undergoing activation.

It will be appreciated that a method of eliminating T-cell undergoing activation from a population of cells can also be effected by the teachings of the present invention.

Thus, according to another aspect of the present invention, there is provided a method of eliminating T-cells undergoing activation from a population of cells. The method is effected by transforming a population of cells, including T-cells undergoing activation, with the nucleic acid construct of the present invention constructed for expressing a cytotoxin. Such a cytotoxin can be, for example, an antisense RNA molecule or a polypeptide which function in disrupting vital cellular functions or initiating apoptosis.

Since the promoter sequence of the nucleic acid construct of the present invention is only activated or upregulated in T-cells undergoing activation, expression of the cytotoxin is restricted to these cells thereby enabling the selective elimination of T-lymphocytes undergoing activation from the population of cells.

It will be appreciated that the suppression or elimination of T-cells undergoing activation can be utilized to treat individuals suffering from immunologic disorders such as autoimmune disorders and the like. In addition, suppression or elimination of T-cells undergoing activation can also be utilized to decrease graft rejection or graft versus host disease (GVHD).

Alternatively, in cases where enhancement of an immune response is desired, such as the case when an individual is infected with a virus or is suffering from a tumors growth, the nucleic acid construct of the present invention can include a polynucleotide encoding a cytokine, such as for example, a lymphokine such as, IL-2, IL-4, IL-7, IL-9, IL-10 or interferon-γ.

When expressed in T-cells undergoing activation, such a cytokine increases the proliferation of T-cells undergoing activation which in turn increases the proliferation of other immune cells regulated by T-cell activation thereby enhancing the immune response.

The nucleic acid construct of the present invention can also be utilized to identify and/or isolate T-cells undergoing activation from a population of cells.

Thus according to another aspect of the present invention, there is provided a method of identifying and/or isolating T-cells undergoing activation from a population of cells. The method is effected by transforming the population of cells with the nucleic acid construct of the present invention constructed for expressing a reporter molecule, and identifying and/or isolating cells from the population of cells which express the reporter molecule above a predetermined background value.

Such a reporter molecule can be an RNA molecule, the expression level of which can be measured via polymerase chain reaction (PCR) or riboprobes. Alternatively and preferably the reporter molecule is a polypeptide such as but not limited to an enzyme, a ligand, a selection marker, or a fluorophore (e.g., green fluorescence protein, GFP), the expression level of which can be measured via biochemical reactions, selection media or fluorescent excitation.

It will be appreciated that the method of identifying and/or isolating T-cells undergoing activation from a population of cells according to the teachings of the present invention, can be utilized, for example, to study factors which influence T-cell activation or to isolate specific subsets of T-cells useful in immunotherapy.

Thus, the present invention provides a novel promoter sequence which can be utilized in expression constructs designed for regulating the immune response mediated by T-cells. Due to its tissue specificity, the promoter sequence of the present invention can be utilized to drive the transcription of regulatory molecules in T-cells undergoing activation and as such to enhance, suppress or eliminate this specific subset of T-cells thereby enhancing or suppressing an immune response.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et at., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

Cloning and Sequence Analysis of Fugu LCK:

A genomic fragment of the Fugu lymphocyte kinase (LCK) was amplified by the polymerase chain reaction (PCR) using degenerate primers 5′-TGY AAR ATH GCN GAY TGY GG-3′ (SEQ ID NO:1) and 5′-GCY TCN GGN GCN GTC CAY TT-3′ (SEQ ID NO:2) complementary to the human LCK exon 10 encoded polypeptide region: CKIADFG (SEQ ID NO:3) and exon 11 encoded polypeptide region: KWTAPEA (SEQ ID NO:4) respectively. The resulting PCR product was cloned into pBluescript and sequenced. A Fugu genomic cosmid library (constructed in LAWRIST 4 by Greg Elgar, UK HGMP Resource Center, Cambridge) was screened with the Fugu LCK fragment and two positive overlapping cosmids, 88A8 and 52C1, were isolated and sequenced. The sequences were obtained by a combination of “shotgun” sequencing and primer walking on an Applied Biosystems 377 automatic DNA sequencer. Homology comparison to database sequences was conducted by using the nonredundant protein database of the National Centre for Biotechnology Information. Coding sequences were identified by their homology to known genes in the protein database, and exon-intron structure of the Fugu LCK gene was confirmed by RT-PCR using total RNA extracted from the Fugu kidney.

Preparation of LCK Promoter-GFP Constructs:

Different lengths of the Fugu LCK promoter that included 84 bp of the first exon were amplified by PCR using specific primers to which restriction sites for KpnI were added at the 5′ end. In addition, the distal and proximal promoters of the human LCK were amplified using specific primers engineered to contain the restriction site for KpnI. PCR amplification was performed in a total a volume of 100 μl containing 20 ng of Fugu or 50 ng of human genomic DNA, 20 mM Tris-HCl (pH 8.4), 50 mM KCl, 2 mM MgCl_{2, 200} mM of each deoxyribonucleotide triphosphate, 25 pmol of each primer and 5.0 units of Taq polymerase (BRL Life Technologies, Gaithersburg, Md., USA). The sequences of primer pairs used to amplify different lengths of the Fugu and human LCK promoters are shown in Table 1 below. The thermal cycling conditions were as follows: denaturation at 94° C. for 1 minute, annealing at 55° C. for 30 seconds and extension at 72° C. for 2 to 4 minutes, repeated for a total of 30 cycles. PCR products were electrophoresed in 1% agarose gels and the band containing the PCR product was excised from the gel and purified using a Qiaquick gel extraction kit (Qiagen Gmbh, Hilden, Germany). The purified DNA was digested with KpnI and cloned into a predigested pEGFP-1 vector (Clontech, USA) upstream of the enhanced green fluorescence protein (EGFP) sequence. Recombinant clones were screened for the presence and orientation of the insert by sequencing with the GFPSEQ1 primer (Table 1).

TABLE 1

Primers list (Restriction enzyme sequences are underlined)

| | | |
|---|---|---|
| SEQ | | |
| ID | | |
| NO: | Description | Sequence |

| | | |
|---|---|---|
| 5 | PF4LCK: forward primer for 2 kb | 5′ GGGGTACCAAGCGCAAGAACACTTCAGG 3′ |
| | Fugu LCK promoter | |
| 6 | PF3LCKS :forward primer for 4 kb | 5′ GGGGTACCAAGCGCAGGCGATTATTATG 3′ |
| | Fugu LCK promoter | |
| 7 | PF2k forward primer for 6 kb | 5′ GGGGTACCATGCTCTCCTCCTCTCACCT 3′ |
| | Fugu LCK promoter | |
| 8 | PRINIS: reverse primer for 2, 4 and 6 kb | 5′ GGGGTACCATGGGACAGTTGCAGTGTTC 3′ |
| | Fugu LCK promoter | |
| 9 | HDF1: forward primer for human distal | 5′ CCGGTACCGAACTCTTGCCCTACTCTCC 3′ |
| | LCK promoter | |
| 10 | HDR1: reverse primer for human distal | 5′ GGGGTACCGTCCTCCTGGCCTAACCTGG 3′ |
| | LCK promoter | |
| 11 | HPF1: forward primer for human | 5′ GGGGTACCGGGGCTTCAAAGTTGAGGGC 3′ |
| | proximal LCK promoter | |
| 12 | HPR2: reverse primer for human | 5′ GGGGTACCGGATAATGGCAGTTCTCACA 3′ |
| | proximal LCK promoter | |
| 13 | GPPSEQ1: primer for sequencing | 5′ CTCCTCGCCCTTGCTCACC 3′ |
| | promoter-GFP constructs | |
| 14 | Forward primer for EGFP gene | 5′ ATGGTGAGCAAGGGCGAGCTAG 3′ |
| 15 | Reverse primer for EGFP gene | 5′ ACTTGTACAGCTCGTCCATGC 3′ |
| 16 | PP4LCKE1, 28 mer, forward primer for 2 kb | 5′ CGGAATTCAAGCGCAAGAACACTTCAGG 3′ |
| | Fugu LCK promoter-GFP construct | |
| 17 | PEGFPRV4, 28 mer, reverse primer for 2 kb | 5′ CGGAATTCGCTGATTATGATCTAGAGTC 3′ |
| | Fugu LCK promoter-GFP construct | |

Transfection into Cell Lines:

JurkaT-cells (human leukemic T-cell line) were grown in RPMI 1640 medium supplemented with 10% FBS, sodium pyruvate, glutamine, penicillin and streptomycin. CHO (Chinese Hamster Ovary), Hela (human cervix adenocarcinoma), Huh7 (human hepatoma), HT29 (human colon cancer), Vero (monkey kidney) and C2C12 (mouse myoblast) cells were grown in DME medium with same supplements as RPMI 1640.

The mixture for each transfection was prepared in a 24-well tissue culture plate as follows: 2 μg of LCK promoter-GFP construct or the Fugu cosmid 52C1 DNA was diluted in 100 ml Opti-MEM (GibcoBRL) and mixed well with 8 μl (16 μg) of DMRIE-C (GibcoBRL) in 1 ml Opti-MEM. The mixture was incubated at room temperature for 30 minute and overlaid with (for 70% confluent adherenT-cells i.e., CHO, HeLa, Huh7, HT29, Vero and C2C12) or resuspended (for 400,000 JurkaT-cells at exponential growing stage) in Opti-MEM washed cells. The cells were incubated for 4 h at 37° C. in a CO₂ incubator, 1 ml of growth medium containing 20% FBS was then added and the incubation continued for 24 to 72 hours. Expression of GFP in the transfected cells was determined using fluorescence visualization in a Leica DMIL inverted microscope fitted with FITC filter.

Generation of Transgenic Mice:

Transgenic mice were generated using standard procedures. Briefly, fertilized one-cell stage mouse eggs isolated from superovulated FVB/N mice were microinjected with purified linearized 52C1 cosmid DNA or with purified LCK promoter driven GFP constructs in which the plasmid backbone was removed by restriction digestion and gel electrophoresis. The Fugu cosmid 52C1 was linearized by digesting with an enzyme that had a unique site in the cosmid. Transgenic founder animals were identified by PCR analysis of genomic DNA isolated from tail biopsies and mated with wild-type FVB/N mice to produce independent lines.

RNA Isolation:

Total RNA was isolated from fresh or frozen tissues of transgenic and wild-type mice by using TRIzol Reagent (GibcoBRL).

RT-PCR:

A reverse transcriptase reaction cocktail containing 0.5 μl oligo(dT)¹²⁻¹⁸, PCR buffer, 2.5 mM MgCl_{2, 0.5} mM dNTP, 10 mM DTT and 1 μl of 10 units/ml of RNase free DNase I (Boehringer Mannheim) in a final volume of 20 μl was added to 3 mg of each RNA sample in DEPC water. The mixture was incubated at 37° C. for 30 minutes and then the Dnase 1 was denatured at 75° C. for at least 5 minutes. The sample was chilled on ice for 1 minute and then prewarmed at 42° C. for 5 minutes. One ml (200 units) of SuperScript II RT (GibcoBRL) was added to the sample which was incubated for an additional period of 50 minutes. The reaction was terminated by incubation at 70° C. for 15 minutes and chilled on ice for 1 minute. Following chilling, the sample was incubated with 1 μl (2 units/μl) of E. coli RNase H (GibcoBRL) for 20 minutes at 37° C. and then used as a template for PCR reactions. Thermal cycling conditions for the RT-PCR were as follow: 95° C. for 30 sec, 55° C. for 1 minute and extension at 72° C. for 45 sec for a total of 35 cycles using DyNAzyme™II DNA polymerase (Finzymes OY, Finland). Fugu LCK expression in mice was detected by using the following primers: 5′-GGC ATC CAC AAC AAC GAG AGG-3′ (SEQ ID NO:18) and 5′-AAG GTA GTC CAC CAG ACT GCC-3′ (SEQ ID NO:19) which correspond to exon eight and nine of Fugu LCK gene respectively. GFP expression in the transgenic mice was detected by using the following primers: 5′-ATG GTG AGC AAG GGC GAG GAG-3′ (SEQ ID NO:20) and 5′-ACT TGT ACA GCT CGT CCA TGC-3′ (SEQ ID NO:21) which amplifies the full length coding sequence of GFP.

Northern Hybridization:

Northern blotting and hybridization were carried out according to standard procedures (Maniatis et al., Ibid.). About 40 μg of total RNA was fractionated on a denaturing gel and transferred onto a nylon membrane and the full length coding sequence of GFP, as amplified by PCR, was used as a probe. The probe was radiolabelled using High Prime labelling kit (Boeringher Mannheim, Germany), according to instructions supplied by the manufacturer.

Western Blot Analyses:

Western blots were performed according to instructions supplied by the ECL Western Blotting Analysis kit (Amersham, England). A monoclonal antibody against GFP (Clontech) was used to check the expression of GFP.

### Example 2

### Experimental Results

Structure of the Fugu LCK Locus:

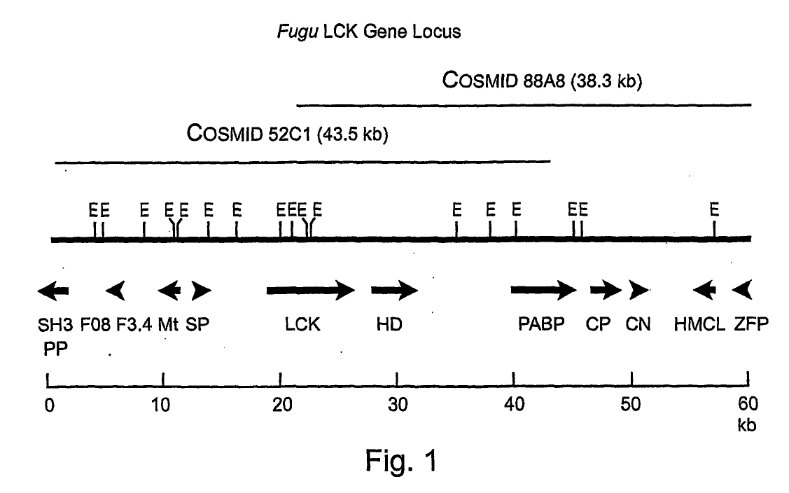

A contiguous DNA sequence of 61 kb was obtained from two overlapping cosmids (52C1 and 88A8, FIG. 1). In addition to containing the sequence of the Fugu LCK gene, this locus contained nine other complete genes: an SH3 philo-protein gene, a gene with homology to C. elegans F08 F3.4 gene, a myotubularin gene, a gene with high homology to a human hypothetical protein gene, a gene for histone deacetylase, a poly A binding protein gene, a cyclophilin gene, a connexin gene and a gene for HMG CoA lyase. The Fugu LCK gene (SEQ ID NO:22) is encoded by 12 exons similar in structure to the human LCK gene. The Fugu LCK gene codes for a protein of 502 amino acids (SEQ ID NO:23) which is 67% identical to the human LCK. The intergenic region between the Fugu LCK gene and it's 5′ upstream neighboring gene sequence includes the complete promoter of the Fugu LCK gene. As such, various length fragments of this sequence (SEQ ID NOs:24-26) were utilized to test expression of Fugu LCK or GFP in cell lines and transgenic animal.

Expression of Fugu LCK in Cell Lines:

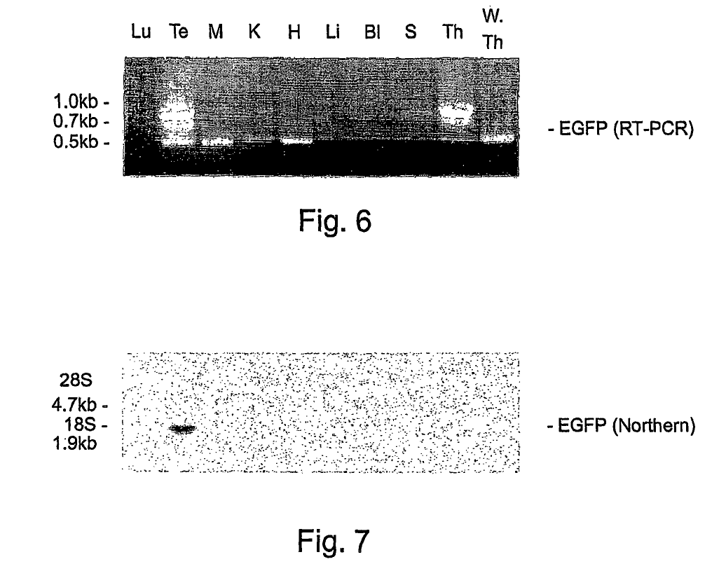

The Fugu cosmid 52C1, which contains the complete sequence for the LCK gene, was transfected into Jurkat and CHO cell lines by electroporation. The expression of LCK was analyzed by RT-PCR. A PCR fragment corresponding to the size of the spliced transcript of Fugu LCK was detected only in JurkaT-cells which are derived from T-cells. The PCR fragment was sequenced to confirm its identity (FIG. 6).

Expression of Fugu LCK Promoter Driven GFP in Cell Lines:

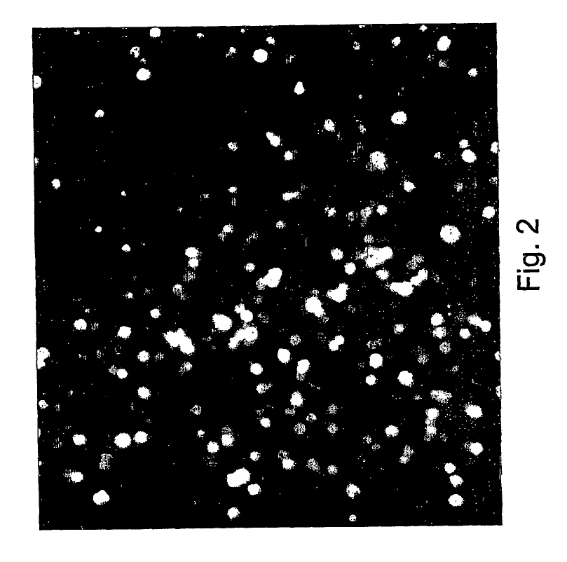

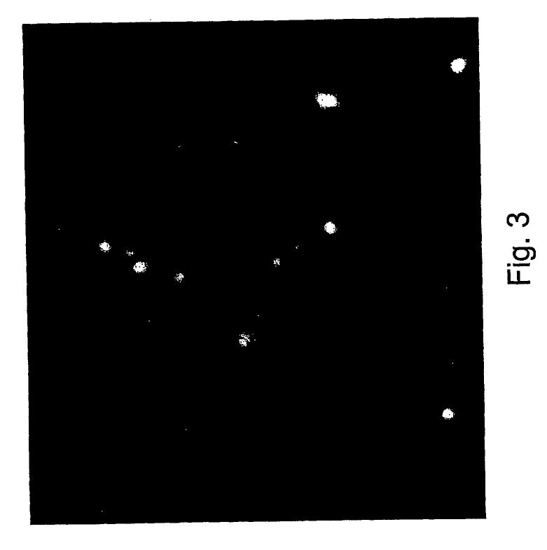

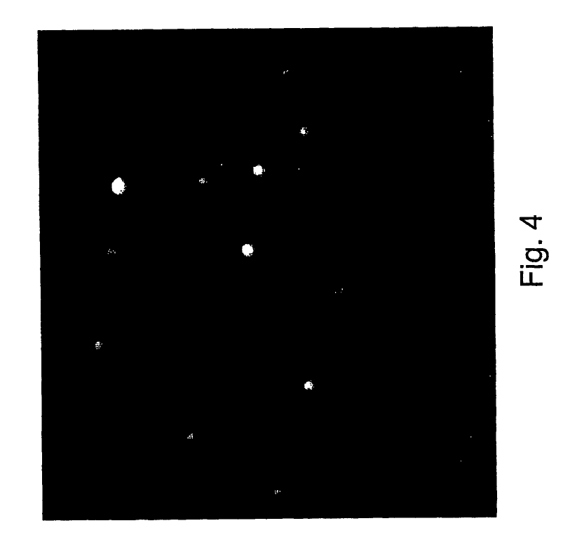

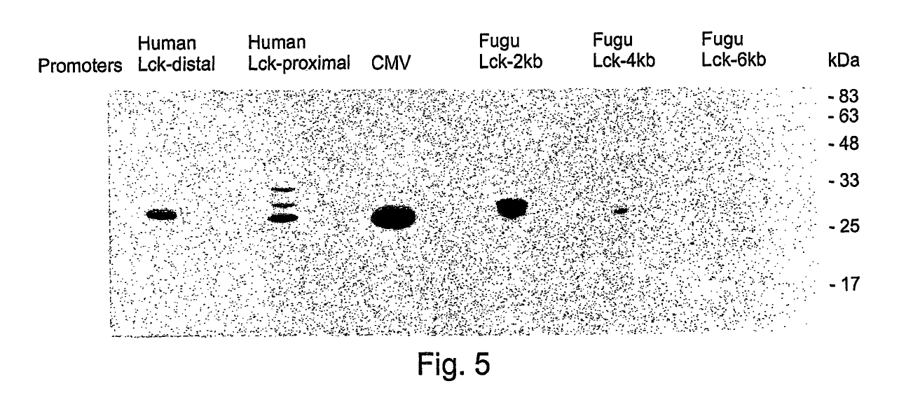

The results from transfection studies of Fugu LCK constructs into various cell lines are summarized in Table 2 below. Visualization of fluorescence revealed that the GFP sequence linked to 2 kb FIG. 2), 4 kb (FIG. 3) or 6 kb (FIG. 4) fragment of the proposed Fugu LCK promoter expressed only in JurkaT-cell lines.

TABLE 2
Expression of GFP-promoter constructs in various cell lines

| | | Fugu | Fugu | Fugu | Human | Human |
|---|---|---|---|---|---|---|
| | | LCK | LCK | LCK | LCK | LCK |
| | CMV | 2 kb | 4 kb | 6 kb | proximal | distal |
| Jurkat | + | + | + | + | + | + |
| CHO | + | − | − | − | | |
| HeLa | + | − | − | − | | |
| Huh7 | + | − | − | − | | |
| HT29 | + | − | − | − | | |
| Vero | + | − | − | − | | |
| C2C12 | + | − | − | − | | |

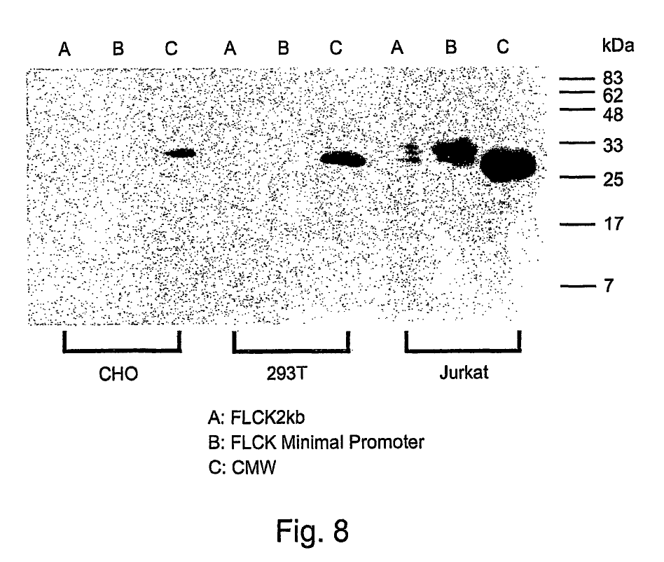

Further, a 914 bp minimal promoter of Fugu LCK was made by deleting 1330 bp of sequence from the 2 kb. A GFP sequence linked to this minimal promoter showed expression specifically and at high level in Jurkat T-cell line (FIG. 8) demonstrating the capacity of this minimal promoter to direct gene expression in human T cells.

Transgenic Mice Bearing Fugu LCK Cosmid:

Injection of the 52C1 LCK cosmid into mice eggs resulted in the generation of 7 independent founder animals carrying the cosmid, of which 5 showed germline transmission of the transgene. F1 and F2 offsprings from these founders were used for assessing the expression of Fugu LCK gene by RT-PCR. The results of these analysis are shown in Table 3 below.

Expression of Fugu LCK was found in the transgenic mouse blood, thymus, spleen, skeletal muscle, heart, and gonads (ovary and testis).

TABLE 3
Expression of the Fugu LCK gene in transgenic mice as detected by RT-PCR

| | Tissue type | | | | | | | | | |
|---|---|---|---|---|---|---|---|---|---|---|
| Transgenic | | | | skeletal | | | | | | thymus |
| line | liver | brain | kidney | muscle | heart | ovary | testis | spleen | blood | thymus |
| 6.19 | − | − | − | + | + | + | | − | + | − |
| 51.16 | − | − | − | + | + | | + | + | + | − |
| 40.46 | | | − | | | | | | | + |
| 44.8 | | | − | | | | | | | + |

Transgenic Mice Bearing Fugu LCK Promoter Driven GFP Constructs:

The 2 kb LCK promoter driven GFP construct injection resulted in the generation of two founders that displayed germline transmission, while the 4 kb LCK promoter driven GFP construct injection produced 6 founder transgenic lines of which 3 displayed germline transmission. F1 and F2 animals generated by mating these founders to wild-type FVB/N mice were used to analyze the expression pattern of GFP.

The results of GFP expression linked to the 4 kb Fugu LCK promoter in the transgenic mice as analyzed by Northern blot analysis are shown in FIG. 7. The 4 kb LCK promoter directed a high level of GFP expression in the thymus and testis, and a lower level of expression in the spleen. Results of Northern blot analyses indicated that the 4 kb Fugu LCK promoter sequence directed expression only in the thymus and the testis (FIG. 7). The 2 kb LCK promoter directed a low level of expression in the thymus in addition to the expression in the testis, but none in the spleen.

### Example 3

### Fugu LCK Minimal Promoter

Deletion studies were performed in order to characterize the functionality of sub-regions of the 2 kb Fugu LCK promoter. Such studies were specifically aimed at identifying minimal regulatory sequences capable of specifically driving gene expression in T cells. Identification of such minimal regulatory sequences was highly desirable since a critical limitation frequently encountered in gene expression systems is the maximum length of the combined regulatory and structural gene sequences which can be employed. Thus, identification of shortened minimal regulatory sequences confers the critical advantage of permitting the expression of structural gene sequences of correspondingly increased length.

As described hereinunder, a 914 bp Fugu minimal LCK promoter (SEQ ID NO:27) which was obtained by deleting 1330 bp from within the 2 kb Fugu LCK promoter was capable of directing specific and high-level reporter gene expression in Jurkat cells.

Results:

Western blot analysis of EGFP expression under the regulatory control of the minimal 0.9 kb Fugu LCK promoter (SEQ ID NO:27) demonstrated specific and high-level reporter gene expression in Jurkat cells. These results, therefore, demonstrated that this minimal promoter is competent to drive efficient and tissue-specific heterologous gene expression. As such, these minimal regulatory sequences represent a further improvement over the 2 kb regulatory sequences described hereinabove. Namely, this 0.9 kb minimal Fugu LCK promoter (SEQ ID NO:27) will permit the expression of gene sequences 1.1 kb longer than those which can be expressed by the 2 kb promoter in the context of gene expression systems limited by the total length of the combined regulatory and structural gene sequences which these can employ. Furthermore, and regardless of such a limiting context, the use of shortened regulatory sequences for expression of a gene of given length is expected to reduce undesirable genetic perturbations arising from the random genomic insertion of foreign transgenes occurring when genetically modifying cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences disclosed therein and/or identified by a GenBank accession number mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

What is claimed is:

1. An isolated nucleic acid comprising a promoter sequence being transcriptionally functional in a T-lymphocyte undergoing activation and transcriptionally less functional in said T-lymphocyte prior to said activation.

2. The isolated nucleic acid of claim 1, wherein said promoter sequence is at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

3. The isolated nucleic acid of claim 1, wherein said promoter sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

4 A nucleic acid construct comprising the isolated nucleic acid of claim 1.

5. The nucleic acid construct of claim 4, further comprising a polynucleotide sequence being under the transcriptional control of said promoter sequence.

6. The nucleic acid construct of claim 4, further comprising a positive and a negative selection markers for selecting for homologous recombination events.

7. A host cell or animal comprising the nucleic acid construct of claim 4.

8. An isolated nucleic acid comprising a polynucleotide sequence at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

9. The isolated nucleic acid of claim 8, wherein said polynucleotide sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

10 A nucleic acid construct comprising the isolated nucleic acid of claim 8.

11. The nucleic acid construct of claim 10, further comprising an additional polynucleotide sequence being under the transcriptional control of said promoter sequence.

12. The nucleic acid construct of claim 11, further comprising a positive and a negative selection markers.

13. A host cell or animal comprising the nucleic acid construct of claim 10.

14. A pharmaceutical composition comprising an effective amount of the nucleic acid construct of claim 10 and a pharmaceutically acceptable carrier.

15. A method of identifying and/or isolating T-cells undergoing activation from a population of cells, the method comprising the steps of:
(a) transforming the population of cells with a nucleic acid construct including a polynucleotide encoding a reporter molecule being under the transcriptional control of a promoter sequence being transcriptionally functional in T-cells undergoing activation and transcriptionally less functional prior to said activation; and
(b) identifying and/or isolating cells from the population of cells expressing said reporter molecule above a predetermined background value to thereby identify and/or isolate T-cells undergoing activation.

16. The method of claim 15, wherein said promoter sequence is at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

17. The method of claim 15, wherein said promoter sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

18. The method of claim 15, wherein said reporter molecule is an RNA molecule or a polypeptide molecule.

19. The method of claim 18, wherein said polypeptide molecule is selected from the group consisting of an enzyme, a ligand and a fluorophore.

20. A method of eliminating T-cells undergoing activation from a population of cells, the method comprising the step of transforming a population of cells including the T-cells undergoing activation with a nucleic acid construct including a polynucleotide encoding a cytotoxic molecule being under the transcriptional control of a promoter sequence being transcriptionally functional in the T-cells undergoing activation and transcriptionally less functional prior to said activation to thereby eliminate the T-cells undergoing activation from the population of cells.

21. The method of claim 20, wherein said promoter sequence is at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

22. The method of claim 20, wherein said promoter sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

23. The method of claim 20, wherein said cytotoxic molecule is an RNA molecule or a polypeptide molecule.

24. The method of claim 23, wherein said RNA molecule is a ribozyme or an anti-sense RNA molecule.

25. The method of claim 23, wherein said polypeptide molecule is an enzyme or a ligand.

26. A method of enhancing T-cell activation, the method comprising the step of transforming a population of cells including the T-cells undergoing activation with a nucleic acid construct including a polynucleotide encoding a cytokine capable of enhancing T-cell activation and being under the transcriptional control of a promoter sequence being transcriptionally functional in T-cells undergoing activation and transcriptionally less functional prior to said activation to thereby enhance T-cell activation.

27. The method of claim 26, wherein said promoter sequence is at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

28. The method of claim 26, wherein said promoter sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

29. The method of claim 26, wherein said cytokine is a lymphokine selected from the group consisting of IL-2, IL-4, IL-7; IL-9, IL-10 and interferon-γ.

30. A method of suppressing T-cell activation, the method comprising the step of transforming a population of cells including T-cells undergoing activation with a nucleic acid construct including a polynucleotide encoding a molecule capable of disrupting a signaling cascade initiated by the T-cell activation, said polynucleotide being under the transcriptional control of a promoter sequence being transcriptionally functional in T-cells undergoing activation and transcriptionally less functional prior to said activation to thereby suppress T-cell activation.

31. The method of claim 30, wherein said promoter sequence is at least 50% identical to SEQ ID NOs:24, 25, 26 or 27 as determined using the BestFit software of the Wisconsin sequence analysis package, utilizing the Smith and Waterman algorithm, where gap weight equals 50, length weight equals 3, average match equals 10 and average mismatch equals −9.

32. The method of claim 30, wherein said promoter sequence is hybridizable with SEQ ID NOs:24, 25, 26 or 27 under hybridization conditions of hybridization solution containing 10% dextrane sulfate, 1 M NaCl, 1% SDS and 5×10⁶ cpm ³²P labeled probe, at 55° C., with a final wash solution of 1×SSC and 0.1% SDS and final wash at 50° C.

33. The method of claim 30, wherein said molecule capable of disrupting a signaling cascade initiated by the T-cell activation is an RNA molecule or a polypeptide molecule.

34. The method of claim 33, wherein said RNA molecule is a ribozyme or an anti-sense RNA molecule.

35. The method of claim 33, wherein said polypeptide molecule is an enzyme or a ligand.

36. A method of identifying a promoter specific regulatory factor, the method comprising the steps of:
(a) providing a reporter construct including a reporter molecule being under the expression control of a promoter sequence being transcriptionally functional in a T-lymphocyte undergoing activation and transcriptionally less functional in said T-lymphocyte prior to said activation;
(b) incubating said reporter construct with a candidate regulatory factor under conditions suitable for transcription and optionally translation of said reporter molecule; and
(c) monitoring a presence of said reporter molecule to thereby determine if said candidate regulatory factor is capabale of regulating expression of said reporter molecule.


SEQUENCE LISTING (27 sequences)

SEQ ID NO: 1 - DNA, 20 bp, Artificial sequence
  Single strand DNA oligonucleotide
tgyaarathgcngayttygg

SEQ ID NO: 2 - DNA, 20 bp, Artificial sequence
  Single strand DNA oligonucleotide
gcytcnggngcngtccaytt

SEQ ID NO: 3 - protein, 7 aa, Homo sapiens
CKIADFG

SEQ ID NO: 4 - protein, 7 aa, Homo sapiens
KWTAPEA

SEQ ID NO: 5 - DNA, 28 bp, Artificial sequence
  Single strand DNA oligonucleotide
ggggtaccaagcgcaagaacacttcagg

SEQ ID NO: 6 - DNA, 28 bp, Artificial sequence
  Single strand DNA oligonucleotide
ggggtaccaagcgcaggcgattattatg

SEQ ID NO: 7 - DNA, 28 bp, Artificial sequence
  Single strand DNA oligonucleotide
ggggtaccatgctctcctcctctcacct

SEQ ID NO: 8 - DNA, 28 bp, Artificial sequence
  Single strand DNA oligonucleotide
ggggtaccatgggacagttgcagtgttc

SEQ ID NO: 9 - DNA, 28 bp, Artificial sequence
  Single strand DNA oligonucleotide
ccggtaccgaactgttgccctactctcc

SEQ ID NO: 10 - DNA, 28 bp, Artificial sequence
  Single strand DNA oligonucleotide
ggggtaccgtcctcctggcctaacctgg

SEQ ID NO: 11 - DNA, 28 bp, Artificial sequence
  Single strand DNA oligonucleotide
ggggtaccggggcttcaaagttgagggc

SEQ ID NO: 12 - DNA, 28 bp, Artificial sequence
  Single strand DNA oligonucleotide
ggggtaccggataatggcagttctcaca

SEQ ID NO: 13 - DNA, 19 bp, Artificial sequence
  Single strand DNA oligonucleotide
ctcctcgcccttgctcacc

SEQ ID NO: 14 - DNA, 21 bp, Artificial sequence
  Single strand DNA oligonucleotide
atggtgagcaagggcgaggag

SEQ ID NO: 15 - DNA, 21 bp, Artificial sequence
  Single strand DNA oligonucleotide
acttgtacagctcgtccatgc

SEQ ID NO: 16 - DNA, 28 bp, Artificial sequence
  Single strand DNA oligonucleotide
cggaattcaagcgcaagaacacttcagg

SEQ ID NO: 17 - DNA, 28 bp, Artificial sequence
  Single strand DNA oligonucleotide
cggaattcgctgattatgatctagagtc

SEQ ID NO: 18 - DNA, 21 bp, Artificial sequence
  Single strand DNA oligonucleotide
ggcatccacaacaacgagagg

SEQ ID NO: 19 - DNA, 21 bp, Artificial sequence
  Single strand DNA oligonucleotide
aaggtagtccaccagactgcc

SEQ ID NO: 20 - DNA, 21 bp, Artificial sequence
  Single strand DNA oligonucleotide
atggtgagcaagggcgaggag

SEQ ID NO: 21 - DNA, 21 bp, Artificial sequence
  Single strand DNA oligonucleotide
acttgtacagctcgtccatgc

SEQ ID NO: 22 - DNA, 7430 bp, Fugu rubripes
atgggatgcaactgcagctcggactattccgacggcgagtggatcgagaacctggatgagatgtgtgaacactgcaactgtcccatagctccccagtcctgcaatccagtgagtgtctacgcacgttcgggggagtcaatgtgagcgtcggctgctcgtgtgcgtcgcgcctcatgatgcaccacgatgggaaaagcagccgtcgcgcgtctggaaaaaaaagtcctggatcttctggctagaatgtctttgaaatgtttgattcaaaccttttgagaactcatccagcatgttctctccccagtacacagatcagctgattcccattcactccccccagctgtcgcctccttcctcgccattaccaggtaatgacacgctttcaaaattcacttttgcttcacatcctccatcagatgggacaaaagccgccacttaaaccctaaagaatccaaggcgatgtgtgcagaactgtagctgcgatgttgcatttaacgatcagcctcatcacgaactcacccctgcgagctttaactgcagtcatttttcaatacggcaccagaattcaaacacagtgggcgcaagataaacactggttctatttcagtcacgcctgtaagcgctcccgaatgcatttcacacatgtttcaaatcattttaatactattgttgtcagacaacctggtggtggccatatacagctatgagcccaagcatgacggcgacctgggcttcgagaagggagacaagctcaagatcatcagcaagtaagctgccgattagcaccttagcatcaactccacgaacacaaatgtctttgatcctaatccctccgcaggcccgcaggcatttgagctccaacatgtttggggtcgtcctcgctgggcggaggcagaactcaactctcagactgaacaattgttgcttatcaggcctgtttgctgtctgctcgtgtttcctgcgtgtctctattttctcctcacggacaatagggaaaaggggcttgggcaaatgacggtgtaggtttaaaggagtagagctgtaaatgtaacagcagcttctcggaggtctgaggttcaacgttatgtgcgcggtggttgctgtctctggggcgtccgtcggacagctacagtgaaacacggctgatatgtaaataatcatggcgctgctgtcgtgtgtttgctacctcagggaggatccggagtggtaccttgcagaatccctcaccactggccagcggggatacgtcccctacaacttcgtcgcaatgtccaccatggagatcgaaccgtacgtcatcacgccgatccccggctctcaccgcgcacgcgtgggtgtcggagggaggggatgtggttcgctcccagtgtggatcacgctgtgtgcttctgctgcaggtggttcttcaagaacatttctagaaatgaagccaacaggcgcctcctggctcccgggaacacgcagggctccttcctgatccgggagagtgaaacgaccccaggtgaacctcatgcacaacaggggagcctggaaaccagttccagtgtcctttagcatcagactgggttaatcctgcctcctaaagatgacttgcttccccatgaaccacttcctgtttcctgtatcacttcacgccctcagtttcactgtggcttttgctgcaattctacattctttgttcgtttaaagtagctgcaggcgttagggtattaaaagcaaaccgaattccccaaactagccaaactattgtgtaactgtggaactgttgtctgcaaagaaatcatccttgtctctgatttcatttgcacccatctccattttagggtcctactccttgtccatcagggacctcgacagcaacgtcggtgacgaagtcaagcactacagaatccgcaacatggataacggcggcttctacatcacggccaagatatccttcaacgctctgaaggaactggtccagcattactcacgtcagtacggcttcacgggggcattaatcaggggcctccatcgtgactgattcccaacccaccctctctccaggtgactcggatggcttgtgcacaaagctggtgaagccgtgccagtccaaagctccacagaagccctggtggcaggacgagtgggagatcccccgcgagtctctgaagctggagcgcaaactcggagcgggacagtttggggaggtctggatgggtgagtggcgcagagatagatgaggaactggcccagttaatgtgagattaaaggttccccatttcagcatgaggagagtgaacagcttgtgctgtgggttcggctgcgtgaccgttcaaaggtttttaaaccgggctttttctgcagcgtgtgcggaacggtttcctccatgcagcctttaattgcaggtttcagtcggttaacagaagagacagaacccctattacgcaatagcacccaaactccacgaatcagcatctcgcagaaagttctaccgagtttcccagatgcataaagttgaacaatgctgaaaaacatgctgacattacgcggataacgaaataatctgccccagttcaacctccaaaacaggccagtccattaacgctggactgaagcacgtttcatagccgctctctatttacagaaccgccccgctcttacaatacattctgggcaccgtatttgttccttaatccaaacactgggtatatttgaataactggagcaacgtctgtttgctctgtttgttctgttgcacggcggtcggccttctctggagaggccgtttcgctccatggagaaggggagggatggtgggaccagaggctcccagtgtgctcccagcattgctcaactctggttttacacgtctggatatgaaaccggttaggttcggtcaggttggcacagctcactggtgtgttattgcgccagagctggaaacgatcgcggtttaacggcagtcacatggatctcctgcttgcttaaggagatatttgatcacggctcctgatatactgagcgcggaagctttacaagctgaggctgaaggctcatcctgttccgcttcacatgacaaatttgttcttgagggagggaaattggtgaccacaacagggaagcattaaagttggtgcgcaggagagggacaccagaaggatgatcccatagttttgctgcatttctgaggccgatctgtatgtttagaggcgtgaatctgtcggttttgtttctacacccagagtccgtgcaccccaacagcccaattccagtctttactctttgatatctgctgtcactcaaatcagtttagtcacttctcctttcaaaatggaggggaaatccttgaaagacagctaaacctacgccgcctctgcgaatggcgccacctgcaggagaattcccttctgatatgtgggtgcaaagtgcctcccccccaacctgttgcaaacgtgcggcagctaaactcggctgcgttaaatcaactcacagctgctctcttcagccaaatgtccttctggtgttttcaggcatccacaacaacgagaggagggtggccatcaagtgcctgaagattggcacgatgtcggtggaagcattcctggctgaggccaacatgatgaagagcctgcagcacatgcacctcgtccgcctcttcgctgtggttacccaggagccgatcttcattgtcacagagtacatggaaaatggtgggtgcaagcactcagctgccaccctcagaccttcagctgggaattccagatattatgattaaaagttcaggaacttctcctcagagggtggttccctgaaatgagctgctagtttttctgaaatgagtccacctgtgagtgtgccagcgccagccggtttcgctgagtcacataaatgtgtctacgcttgtctggagccatcgttttctcgatctgccctttccctctaacccccaaagcagctccgcctggtgatctctgcaggggtacagcagagtcaggatcgaacatgtcgaggccagatcagtgtgtttcacctcttgagtcacggtgctttcagacgccaaccgcttccctccttctgacaaatttgccatttcctctcatcgctctgcctttccctccccgggttacgtgagtttgatgaataacacgatgacccgtgacctgtattatgccgccgtgcgcgccggcctaaattagggagcaaactcctttgcctcgcctcccaaagcacgctgtcccgctgaagtgctcgggctattttcgtcctgtcctcatcgccctcctctgttcgccgctccgtgtgaaaaagcaagcgtgaccttcgactctctctccctggtcaggttgtcagtgtttgcctgccgagacggaagtctctaccgcgctgtctgttgtgagacggtgggtgggcagttaaggcggaggaggtgattcaccgattattgttcagcgccgatggaacctggctagcttccacggtcgggcacatgcagaatgttgcagtggtaaacaatcctcagacaatagcagtctggcatcaggccactccgttttgctcattgctcgttgactttctaaaacacaaaaacaccagcaaattttcaccggggaaccgcagacgcgagaggtgacagattctgaaaagagtgcggggtgctcttggtcgccaaggcagcagtggcacagaagggatctggtttctgtttgtccgaggaagacgcataaaacgcaggtccctcacagagcagatggcatccagctctttgtctgggccgggacgggacaacaaacgtggcctttgtggtcgggaggaacgatgcctcagagttcctgtctcttcagtttgtccccttttttattatcacagataacaaagctggaaaataacaggcgtgtcggcccggctttcctttgtgaccttgacagggagcttttcccccagctccctgtcttcaaaaagggtcttcctggtttcaggccggggagcaaaaggcctcatcttccttggcacagtcggtcctcttatggaatttccttcggactttgggcacaaacacgcagatgtattgttcctcgctaatcagcagaaagtcgtgtgcacgtcgtcgtgactgatggttcctgcctggctgtggcctgaaatgagctgctcttctgctccggtgtggtttatttacgtccctgggaaatctgtcacccttaaacacagcttttatttgacgtgatcataaattgaaggtcgttttctttcaggcagtctggtggactaccttaaaacaacggagggaagcagtttgtccataaacaccctgatcgacatggcatctcaggcaagacgtctctttcttttacatttcctttattgtcttccttttccctcagcgtggtcctcgccgtcaggaaggtgtcactgctaatctttgcaaaacaggattttgtgcgtttcatcttccgtcatctaatgcagacgtagctaaaccaccgtgaaagtgaataaatgagttagcaactaaaaccacagataagttcctttacatcttaaactgagagatgaccttttcaggttttgtattggcaattctagtgttacctaagcttcaaacctggaaacagcctggcacacacctccttgtccttctatctttgtgaggactttcatgcatgcattgtattccccagcacctaactgaccctaaccagccccaccaataccttgaccctaactctgacccaacccaactctaaccccaaccttcaaaccaagtcttaacccttaaacagtcctttgaagttgtgaagaccggtcaaaatgtcctggctagtagacagaggattttagcagtcagtatgtagcaagtgcaagaacacacacacacacacatacagttgaacgagtttagtggctttggctcctttatttaagattaagattaagattaagatttgagataatagatgagaactggatgatttttattgtgagcaaatgcaaatccacgttttatctctagtgtttagaacattatgtaacagcgtggcttcctgggtttcgtcttttagcagtgtatgggtcactaaccatttaaaagttacaagttcactcctaaaattgacaagttttgattgtttttacgttatttttacaatattttataatgaattgcagtcatggccacagctcatgtaaaagagcaactttaacttgcagtacatgtaggctagggtaagtgtgggaatggaacacacaaccacaggaagacaaaagagtttctatgcacgtcaccttggtgatgatgtctataactgagaggaagtgattttacaaaagcaacctaggttacaattccttttctaaaggtataaagcttagtaaaaacccgtgtatgcctaataacttaaaacgctgatggaaaagagctccaaaacggaatgtgaaagcaaatattgcaaaataatgattaatatagtgatttattatctatgttatttatattgtggagggtaaagaagtgtaaaggtcgcagtataagtgaaaaacacgtcccactcgtacgtaaatctcacatttctgacgagagcccggggcccctcggccctgagtgtcacatttttctggtatgtagctgaacatctgacccaacacaatattcatacagaagcaacagtgaatttcccctgacagtcacattaatcatcttgtaaggaggtggtcgatttccttttgcctcttgattattcagaacccaaatgtgccctttaacaaatcccttattttaacatctgttgcctgtctgacgctgtgctgccccctacaggtggcggatggaatggccttcatcgaggcgaggaattacatccatcgcgacctgcgagcagcaaacatcctggtgtctcatgaactcatctgcaagatcgcagactttggcctggcgaggctcatcgagaacaacgagtacacggccagagaaggtaaccagagggagcggataacaggtttaactgaggaagagtccgaatctcacacgccgctttcacgcttgcttttctgcttccaggtgctaaatttcccatcaagtggaccgccccagaggctataaactacggcaccttctccataaaatcagatgtgtggtcatttgggatcctcctgacagaaatagtgacatatggacgcataccttaccctggtaggtgacttttctttgtccaaacccgttcattcttaagatcactcactactcactcaatatactccttatgtgtccttcagggatgtccaacccagaggtcatacatcagctggagcaaaactacaggatgccaaagccggagaactgtcctgacgggctctacaacttcatgctgctgtgctggagggaaaaaccagaggacagacccacatttgactacctgaggagcgtcctggaagacttcttcaccgccactgag

SEQ ID NO: 23 - DNA, 2450 bp, Fugu rubripes
aagcgcaagaacacttcaggcgcccacatttcaacctgcagcgtctgaagtcaaacgctttcatggaaattgagtcagctcaggcggtttcattgcaagtaacacacagtacgctgcaagaggcttcaaaacgcacctatatgtgcacttcctgtgatgctaggaccaacttcctgtcagtcagtgcaggggtgcgagtagacacttgccacaacaggagagaggcaggccactgggtgaacgagtttagagcggtcttgtagagtctaatagcgtctctcttcgtggtcagtgttacgtctctgagctatttttgtccccatttgtgtgtttcgggacagatgtttctggcccccagtcacagacagcaactttgacacatatgacctttgacatatttacttggaggcaggaaaccacccatcagggctggaatggtgcatcagtgtggctttgttgtggaaaataaaaagtaataaataaagcactgcaccgactgctgtgcctgtctggccctgcaccgacggtgccctcatcccaaaggtcacgcacttgcacatgttacccacaagagaggtgcagttggagcccctccgtgcataacggtattggaacattcatttttggtgacgtcctgacccggtaacgttcccccccggctcgtgcacagctgcagcgtgaagcacctgctgaggctagcaaagcaagcctcaacccctcaacgacaggaaatgccccagctcgcactccaccgcgtcgaagcgctgaagatgcagacatttcgttaagacagacaggcagacaatgacctgctggcgcgttggtcgaaaacctacaaacctgtgaaagaaacctttataaaccagtaattacatggcagcgtgcagaatctgacaatattggacgtcgaatatacactttctatctctggaggtaagtttaatgcttgttagactgcataatatagataataaacaggttgtgagtcaatgtgagcgtttatacaatatgtttagtgttaaaattaacactgcttacagaaataatgttaaagatagaagcagctagaaatgactttggtaagggaagcgagaggaaggttagtagcatctatattcacagagaaagcctgataacacaccaaaaaggattcgttttcattctattcgtaggaaatacacaaagtaatgaagctttcataatcagctttcattttatctgtctgaaaaatcatgtcgctgaagacgatggcatcaggatgtgaatggcaaaaaataaataaattacttttctgcagcacttaaaacctaaattttaggtaaatattctacttaaggcattaattaatatgatttaatcactgatcattactttaatgttaagataatgtgtaattactactaaattactaaactctactaaactccatggtagggaatgaggtcatacttgagaggtttaaggtgctgaatatatttcagacgaccccataaaggacaaagaggatccatattatggtaaaattaaaaagaggattcataaatgtttatgattaatacgcaggagtgagacgctccaagaactctttgtaactcgtgagtaacgaatgaaaatagtttcgtagcagttattcattgtcataataattgaaaaatgaattacgccatcgtccaatactttgcagacgtcccccccccccagctgccgaagatatataagaggagcgaccctccgaatggaaaacgctgcctttgtttgtgtgactgagttctggcccggtccgccgaccgtttccagtaaaagaactgactccccatcacgcggcgctcctcgcctcctctggagcgttgttgttgctgagggggtcacggttgccgggtcgggcctcagatttttctcatggctctcagcctttgggacggaccgctggggtgcttagcccagtgacctcgttccacgaggggtccgacgagggccaagagagcttgagaaacacagagaagtctactgtataactttatacagaacacgtgcggatgctggatcaggcaggattagaatcgctgggcttggattcggatttaattgtgtgagcctctgtgattgtcccggctgtgatcagggaggttctttgttctcagtgtgttttctgcagctttaattgaacttttcaggccttccttcttcagctcgggtcaccgccggcctccattttgcgacgaggccccccggtgacatgttcttcacttagaaataaaaaaataaatccccttccttcttttctccctcctgtattttaccgatgcagccataacgcgcttgcgatgcattcggctcttcataatttattttctttcaggaacgatgggatgcaactgcagctcggactattccgacggcgagtggatcgagaacctggatgagatgtgtgaacactgcaactgtcccat

SEQ ID NO: 24 - DNA, 4103 bp, Fugu rubripes
aagcgcaggcgattattatggatttgtgatgcacaatcgtatgtagttgtgatttaaagactgtctgggagcatttgaacacaggaggttttttccactttgacacaaatcagacagaaatagagaagcctccctgcagccctggtagagacgaaaactaacaagaaggagtcgagtgctgcagtaaaaatccagaaatgtatatttttgtatattatgtattttgtctattggttaaaaataaaaaaaactgctctgaacttgtattttcatcttgaatcgtgattattttttaagaatctgctgagatttttccccattaaacgaataagcgatgcattaaatgtgccattcccccataactgtcactcccccttttctccagatgctgaccatctatgttgactaccctgtactttacccggataaggagttttattttggttctcgatgattaaaattgcccatttttccagctctgatcactaattaatgaatcgtttcactacagatgttgcataaggcgttaaagcaactcacacgatatgaatggttccgcttaagtcctgcagccacgttcatgcataatcacattccatcacagtgagcagacatttagttggtcctttcagatcacgccgggcagaaggtcacgttaggtcattttaggagatggtgatcgcccaagtgcatttttgtatttgtgtgcaggagtaaaataaatacatgaaggatgtgcgttagacccagtctggcctgtaaacaacatatttagcacaagtgtgaatgtttgcacataaatgcaacagtttggggtgggactctcatcgctgctcagagcacatgctctggcacatttcccccgtgtcatcccttcaaagccgcatcgacgccaccccgcacaatagtctcctctttccctccgtgacctgctgtaatatccagacatgtgaacggggagccggagggttgcaccgagcggcggacattgtgaggcggagaggagcagaacatgaaaccagacccgcatcggcgcagagggcaactgtgtgaaatgagaactcctcctggactgttccacatttaaggacgtggacggcctcaagaatggaaatggagatcatatggaaaatgcaggactgaggcattccgcctccaggcgcttcttctcagtggatgcttttacaatgaaatgccacagacttgtgtttctcatgccccttgaattcaatgtgaacctttccctacatgataacacgtgattctgtaggagtagaaagtgctgttttactatcgaggcagcctgaatctgttaacagcaacaataaagggcacgcatgaggtgagcgtcaggagaaagagaaggtaggccagcacctgcagtgtgagggaggcagcggaggggtcggaaaaccatctgcacgtcgatggtatcagtccccaaactgcgtaacagaatgtgatttaaaactacatttgggggaggtgtcagctcacagggaaacccaccaaagcggagctgcaaaggtgctcgacgggcccaggtgaccccgcggggtcagccatcactcatccctttaaacctaaaatcactgcagtcccgcggcatcgttggaatcggctgagcggatttggaaaatctaaattgtggaaaagcgcaagaacacttcaggcgcccacatttcaacctgcagcgtctgaagtcaaacgctttcatggaaattgagtcagctcaggcggtttcattgcaagtaacacacagtacgctgcaagaggcttcaaaacgcacctatatgtgcacttcctgtgatgctaggaccaacttcctgtcagtcagtgcaggggtgcgagtagacacttgccacaacaggagagaggcaggccactgggtgaacgagtttagagcggtcttgtagagtctaatagcgtctctcttcgtggtcagtgttacgtctctgagctatttttgtccccatttgtgtgtttcgggacagatgtttctggcccccagtcacagacagcaactttgacacatatgacctttgacatatttacttggaggcaggaaaccacccatcagggctggaatggtgcatcagtgtggctttgttgtggaaaataaaaagtaataaataaagcactgcaccgactgctgtgcctgtctggccctgcaccgacggtgctcctcatcccaaaggtcacgcacttgcacatgttacccacaagagaggtgcagttggagcccctccgtgcataacggtattggaacattcatttttggtgacgtcctgacccggtaacgttcccccccggctcgtgcacagctgcagcgtgaagcacctgctgaggctagcaaagcaagcctcaacccctcaacgacaggaaatgccccagctcgcactccaccgcgtcgaagcgctgaagatgcagacatttcgttaagacagacaggcagacaatgacctgctggcgcgttggtcgaaaacctacaaacctgtgaaagaaacctttataaaccagtaattacatggcagcgtgcagaatctgacaatattggacgtcgaatatacactttctatctctggaggtaagtttaatgcttgttagactgcataatatagataataaacaggttgtgagtcaatgtgagcgtttatacaatatgtttagtgttaaaattaacactgcttacagaaataatgttaaagatagaagcagctagaaatgactttggtaagggaagcgagaggaaggttagtagcatctatattcacagagaaagcctgataacacaccaaaaaggattcgttttcattctattcgtaggaaatacacaaagtaatgaagctttcataatcagctttcattttatctgtctgaaaaatcatgtcgctgaagacgatggcatcaggatgtgaatggcaaaaaataaataaattacttttctgcagcacttaaaacctaaattttaggtaaatattctacttaaggcattaattaatatgatttaatcactgatcattactttaatgttaagataatgtgtaattactactaaattactaaactctactaaactccatggtagggaatgaggtcatacttgagaggtttaaggtgctgaatatatttcagacgaccccataaaggacaaagaggatccatattatggtaaaattaaaaagaggattcataaatgtttatgattaatacgcaggagtgagacgctccaagaactctttgtaactcgtgagtaacgaatgaaaatagtttcgtagcagttattcattgtcataataattgaaaaatgaattacgccatcgtccaatactttgcagacgtcccccccccccagctgccgaagatatataagaggagcgaccctccgaatggaaaacgctgcctttgtttgtgtgactgagttctggcccggtccgccgaccgtttccagtaaaagaactgactccccatcacgcggcgctcctcgcctcctctggagcgttgttgttgctgagggggtcacggttgccgggtcgggcctcagatttttctcatggctctcagcctttgggacggaccgctggggtgcttagcccagtgacctcgttccacgaggggtccgacgagggccaagagagcttgagaaacacagagaagtctactgtataactttatacagaacacgtgcggatgctggatcaggcaggattagaatcgctgggcttggattcggatttaattgtgtgagcctctgtgattgtcccggctgtgatcagggaggttctttgttctcagtgtgttttctgcagctttaattgaacttttcaggccttccttcttcagctcgggtcaccgccggcctccattttgcgacgaggccccccggtgacatgttcttcacttagaaataaaaaaataaatccccttccttcttttctccctcctgtattttaccgatgcagccataacgcgcttgcgatgcattcggctcttcataatttattttctttcaggaacgatgggatgcaactgcagctcggactattccgacggcgagtggatcgagaaccggatgagatgtgtgaacactgcaactgtcccat

SEQ ID NO: 25 - DNA, 6222 bp, Fugu rubripes
atgctctcctcctctcaccttttctcgatggcgtccacgtttctgaggagcagcaggacctgccgggagctgccctccgctcgctccgagaagagcaggtggtggccggtgatacacagcgtccctctgcaaggcgggtgcggcggctgctggaggacaacatcctccacgttggccgtcttgatgtgctcagaaaactccattcctccttttgggcttccctccggaacttctgctaccgacttgaaatcacttcctcgagtctggtgagaaaatgaaagcgggatcccccgtgcgtcctctcggctgggtacgactgcagatgcgctctttctgtcggctgataaggggtctataagtaggcgtttttttctgcgcatagttgaactacgcctgtttccacacatgtggctcagcacgaaagggttctgaaatgtaaacgtgagccacagaagacggaagtgcaagtttcctgtgaggctgacatgtgtcgtgcgttggagggcgaagtttttgaacactacgcaaaagaggaaccagaagtgatgtgatgtgtcatataacaaatggccactaggtggaagcagagatgtggggtgaattcggtatgatgcgcccatacttaatgataataatcggcatgttagccgacataaaaactcatttatcagctataacacgaatctttgccccccacattattcttttgacgcaatttttgtcgtatcaatatgatacagagcaatccacccgtttaaggctccaggtttaaggtcctttatttgtcatatcaatcaacacttgcatgaaaattattacttaggtcccaattataaccaaattaaaagaagtataggcattgaaactaaatggatagagaaaagaagccagtggtatatataaagaaaattgtgtatagtttaaattagaagaaaaatgaattaaaatgtcgcagattcataatcccgacgtttattttaagtaaaagcctcaagcaaacttgaacctgaaaaaaaatgatttactcatttttacaatcattctaaaaacggtcctttatgtcatcgatataatgaggggaaactagtgacctcgctgcttctaagtaacttctaagaaacgatccttcctcaatccggcttccggctgtgtgaaatgggcggtcctgtctgcatataaaggaaggcaagagacctgttgcgctccaagcagccgatcctcacgccggattccccaaagaagtggtcgcctgcgcgcagatgtattttcaagagattggaggcgagtcctcagaaggagagacggaggatctggacagcctgaaagcgctcacggagaagctgaagctgcagacgcgcagaccctcctacctggagtggcaggagcggctccagagtcgaccctggaccgagaacggtgtggactccggacaggtctctgctccccggagaaacaaggacgcggacgtggtggtgggcaacatctgcggcttcaacacgattgacgacgctttggagcatctgaggaaagagctggtgagggaggggtggtggtgcgcgcattccccgtacacctgggcgggatgtcaaacacacaaccctgtacattttactgatacgcacttttcaaagtttccccaccttcgcgtattcctgtatccacagagggagatgcaggtccaggataaccgcctggcccgccagctgatccgcctgcgcggggagatccaccagctgaaggtggagcaggtttgccaccggcacaaggagatgctggacgacgccacgtacgagctgaggagtgcggggaagagtcggatctgctgtgcgacatccccatgaaggcagctttcgccctgtccaccccactcaaacacctgggcctcaccaagatgaacatcaactccaggcgcttctccttgtgttagacctgcccgggtgccagaggtcatgtccacctgtgggcggctgtttggacctggtgtcctcacatgaaaatagcttcttcaggactgcctggtgtggagacgtggggaaacacactgcacatctcctccaggagagtcccggctaccccggctcttcagtgtcaaacagccggagaaaggttaaatcaggaagcgcaggcgattattatggatttgtgatgcacaatcgtatgtagttgtgatttaaagactgtctgggagcatttgaacacaggaggttttttccactttgacacaaatcagacagaaatagagaagcctccctgcagccctggtagagacgaaaactaacaagaaggagtcgagtgctgcagtaaaaatccagaaatgtatatttttgtatattatgtattttgtctattggttaaaaataaaaaaaactgctctgaacttgtattttcatcttgaatcgtgattattttttaagaatctgctgagatttttccccattaaacgaataagcgatgcattaaatgtgccattcccccataactgtcactcccccttttctccagatgctgaccatctatgttgactaccctgtactttacccggataaggagttttattttggttctcgatgattaaaattgcccatttttccagctctgatcactaattaatgaatcgtttcactacagatgttgcataaggcgttaaagcaactcacacgatatgaatggttccgcttaagtcctgcagccacgttcatgcataatcacattccatcacagtgagcagacatttagttggtcctttcagatcacgccgggcagaaggtcacgttaggtcattttaggagatggtgatcgcccaagtgcatttttgtatttgtgtgcaggagtaaaataaatacatgaaggatgtgcgttagacccagtctggcctgtaaacaacatatttagcacaagtgtgaatgtttgcacataaatgcaacagtttggggtgggactctcatcgctgctcagagcacatgctctggcacatttcccccgtgtcatcccttcaaagccgcatcgacgccaccccgcacaatagtctcctctttccctccgtgacctgctgtaatatccagacatgtgaacggggagccggagggttgcaccgagcggcggacattgtgaggcggagaggagcagaacatgaaaccagacccgcatcggcgcagagggcaactgtgtgaaatgagaactcctcctggactgttccacatttaaggacgtggacggcctcaagaatggaaatggagatcatatggaaaatgcaggactgaggcattccgcctccaggcgcttcttctcagtggatgcttttacaatgaaatgccacagacttgtgtttctcatgccccttgaattcaatgtgaacctttccctacatgataacacgtgattctgtaggagtagaaagtgctgttttactatcgaggcagcctgaatctgttaacagcaacaataaagggcacgcatgaggtgagcgtcaggagaaagagaaggtaggccagcacctgcagtgtgagggaggcagcggaggggtcggaaaaccatctgcacgtcgatggtatcagtccccaaactgcgtaacagaatgtgatttaaaactacatttgggggaggtgtcagctcacagggaaacccaccaaagcggagctgcaaaggtgctcgacgggcccaggtgaccccgcggggtcagccatcactcatccctttaaacctaaaatcagctgcagtcccgcggcatcgttggaatcggctgagcggatttggaaaatctaaattgtggaaaagcgcaagaacacttcaggcgcccacatttcaacctgcagcgtctgaagtcaaacgctttcatggaaattgagtcagctcaggcggtttcattgcaagtaacacacagtacgctgcaagaggcttcaaaacgcacctatatgtgcacttcctgtgatgctaggaccaacttcctgtcagtcagtgcaggggtgcgagtagacacttgccacaacaggagagaggcaggccactgggtgaacgagtttagagcggtcttgtagagtctaatagcgtctctcttcgtggtcagtgttacgtctctgagctatttttgtccccatttgtgtgtttcgggacagatgtttctggcccccagtcacagacagcaactttgacacatatgacctttgacatatttacttggaggcaggaaaccacccatcagggctggaatggtgcatcagtgtggctttgttgtggaaaataaaaagtaataaataaagcactgcaccgactgctgtgcctgtctggccctgcaccgacggtgctcctcatcccaaaggtcacgcacttgcacatgttacccacaagagaggtgcagttggagcccctccgtgcataacggtattggaacattcatttttggtgacgtcctgacccggtaacgttcccccccggctcgtgcacagctgcagcgtgaagcacctgctgaggctagcaaagcaagcctcaacccctcaacgacaggaaatgccccagctcgcactccaccgcgtcgaagcgctgaagatgcagacatttcgttaagacagacaggcagacaatgacctgctggcgcgttggtcgaaaacctacaaacctgtgaaagaaacctttataaaccagtaattacatggcagcgtgcagaatctgacaatattggacgtcgaatatacactttctatctctggaggtaagtttaatgcttgttagactgcataatatagataataaacaggttgtgagtcaatgtgagcgtttatacaatatgtttagtgttaaaattaacactgcttacagaaataatgttaaagatagaagcagctagaaatgactttggtaagggaagcgagaggaaggttagtagcatctatattcacagagaaagcctgataacacaccaaaaaggattcgttttcattctattcgtaggaaatacacaaagtaatgaagctttcataatcagctttcattttatctgtctgaaaaatcatgtcgctgaagacgatggcatcaggatgtgaatggcaaaaaataaataaattacttttctgcagcacttaaaacctaaattttaggtaaatattctacttaaggcattaattaatatgatttaatcactgatcattactttaatgttaagataatgtgtaattactactaaattactaaactctactaaactccatggtagggaatgaggtcatacttgagaggtttaaggtgctgaatatatttcagacgaccccataaaggacaaagaggatccatattatggtaaaattaaaaagaggattcataaatgtttatgattaatacgcaggagtgagacgctccaagaactctttgtaactcgtgagtaacgaatgaaaatagtttcgtagcagttattcattgtcataataattgaaaaatgaattacgccatcgtccaatactttgcagacgtcccccccccccagctgccgaagatatataagaggagcgaccctccgaatggaaaacgctgcctttgtttgtgtgactgagttctggcccggtccgccgaccgtttccagtaaaagaactgactccccatcacgcggcgctcctcgcctcctctggagcgttgttgttgctgagggggtcacggttgccgggtcgggcctcagatttttctcatggctctcagcctttgggacggaccgctggggtgcttagcccagtgacctcgttccacgaggggtccgacgagggccaagagagcttgagaaacacagagaagtctactgtataactttatacagaacacgtgcggatgctggatcaggcaggattagaatcgctgggcttggattcggatttaattgtgtgagcctctgtgattgtcccggctgtgatcagggaggttctttgttctcagtgtgttttctgcagctttaattgaacttttcaggccttccttcttcagctcgggtcaccgccggcctccattttgcgacgaggccccccggtgacatgttcttcacttagaaataaaaaaataaatccccttccttcttttctccctcctgtattttaccgatgcagccataacgcgcttgcgatgcattcggctcttcataatttattttctttcaggaacgatgggatgcaactgcagctcggactattccgacggcgagtggatcgagaacctggatgagatgtgtgaacactgcaactgtcccat

SEQ ID NO: 26 - DNA, 914 bp, Fugu rubripes
gccacaacaggagagaggcaggccactgggtgaacgagtttagagcggtcttgtagagtctaatagcgtctctcttcgtggtcagtgttacgtctctgagctatttttgtccccatttgtgtgtttcgggacagatgtttctggcccccagtcacagacagcaactttgacacatatgacctttgacatatttacttggaggcaggaaaccacccatcagggctggaatggtgcatcagtgtggctttgttgtggaaaataaaaagtaataaataaagcactgcaccgactgctgtgcctgtctggccctgcaccgacggtgctcctcatcccaaaggtcacgcacttgcacatgttacccacaagagaggtgcagttggagcccctccgtgcataacggtattggaacattcatttttggtgacgtcctgacccggtaacgttcccccccggctcgtgcacagctgcagcgtgaagcacctgctgaggctagcaaagcaagcctcaacccctcaacgacaggaaatgccccagctcgcactccaccgcgtcgaagcgctgaagatgcagacatttcgttaagacagacaggcagacaatgacctgctggcgcgttggtcgaaaacctacaaacctgtgaaagaaacctttataaaccagtaattacatggcagcgtgcagaatctgacaatattggacgtcgaatatacactttctatctctggaggtaagtttaatgcttgttagactgcataatatagataataaacaggtttaacgcgcttgcgatgcattcggctcttcataatttattttctttcaggaacgatgggatgcaactgcagctcggactattccgacggcgagtggatcgagaacctggatgagatgtgtgaacactgcaactgtcccat

SEQ ID NO: 27 - protein, 502 aa, Fugu rubripes
MGCNCSSDYSDGEWIENLDEMCEHCNCPIAPQSCNPYTDQLIPIHSPQLSPPSSPLPDNLVVAIYSYEPKHDGDLGFEKGDKLKIISKEDPEWYLAESLTTGQRGYVPYNFVAMSTMEIEPWFFKNISRNEANRRLLAPGNTQGSFLIRESETTPGSYSLSIRDLDSNVGDEVKHYRIRNMDNGGFYITAKISFNALKELVQHYSRDSDGLCTKLVKPCQSKAPQKPWWQDEWEIPRESLKLERKLGAGQFGEVWMGIHNNERRVAIKCLKIGTMSVEAFLAEANMMKSLQHMHLVRLFAVVTQEPIFIVTEYMENGSLVDYLKTTEGSSLSINTLIDMASQVADGMAFIEARNYIHRDLRAANILVSHELICKIADFGLARLIENNEYTAREGAKFPIKWTAPEAINYGTFSIKSDVWSFGILLTEIVTYGRIPYPGMSNPEVIHQLEQNYRMPKPENCPDGLYNFMLLCWREKPEDRPTFDYLRSVLEDFFTATERQYQE